(19) United States
(12) Patent Application Publication
(10) Pub. No.: US 2013/0258282 A1
(43) Pub. Date: Oct. 3, 2013

(54) OPHTHALMOLOGIC APPARATUS

(71) Applicant: CANON KABUSHIKI KAISHA, Tokyo (JP)

(72) Inventors: Atsushi Goto, Miura-gun (JP); Kazuhide Miyata, Yokohama-shi (JP); Makoto Sato, Tokyo (JP); Tomoyuki Makihira, Tokyo (JP); Yoshihiko Iwase, Kyoto-shi (JP); Kazuro Yamada, Kawasaki-shi (JP); Ritsuya Tomita, Kawasaki-shi (JP); Yohei Minatoya, Yokohama-shi (JP); Daisuke Kibe, Chigasaki-shi (JP); Hiroyuki Shinbata, Tama-shi (JP)

(73) Assignee: CANON KABUSHIKI KAISHA, Tokyo (JP)

(21) Appl. No.: 13/850,982

(22) Filed: Mar. 26, 2013

(30) Foreign Application Priority Data
Mar. 30, 2012 (JP) 2012-082688

(51) Int. Cl. A61B 3/10

(52) U.S. Cl. CPC A61B 3/1005
USPC 351/206

(57) ABSTRACT

An ophthalmologic apparatus includes an obtaining unit configured to obtain an anterior segment image of a subject's eye, a tomographic image obtaining unit configured to obtain a tomographic image of an anterior segment of the subject's eye, a calculation unit configured to calculate a thickness of a cornea based on the tomographic image, and a display control unit configured to cause a display unit to display the anterior segment image, a closed curve on the anterior segment image, and a smallest thickness of the cornea among thicknesses of the cornea in a portion where the closed curve lies.

### BACKGROUND OF THE INVENTION

1. Field of the Invention

Aspects of the present invention generally relate to an ophthalmologic apparatus.

2. Description of the Related Art

Penetrating keratoplasty includes cutting out a patient's cornea in a circular shape to remove thereof and sewing a donor's cornea cut out in a similar size on for replacement.

A knife, called a trepan, for punching out a central portion of the cornea in a circular shape is conventionally used to cut the patient's cornea. A portion of the donor's cornea is similarly punched out in a circular shape, and sewn on 360° with a thread. The sewing of two corneas by hand, however, has been pointed out to inevitably distort the cornea after surgery and cause astigmatism (irregular astigmatism) that cannot be corrected by glasses. It has also been pointed out that the cornea decreases in strength and the cut can open from a bruise after surgery.

A femtosecond laser can be used to freely cut the cornea. Utilizing this, the application of a femtosecond laser to corneal transplantation has recently been attempted. “Ganka Saishin Shujutsu [Latest Ophthalmologic Surgery],” Vol. 53, No. 10, 2011, Kanehara & Co., Ltd., discusses that cutting to a shape that has not been possible by conventional trepanning (such as a top-hat shape where the epithelial side is cut large and a mushroom shape where the endothelial side is cut large) can be performed to allow excellent healing of the wound and recovery of a nearly normal cornea shape. The degrees of freedom of keratotomy have thus been on the increase in recent years.

It has been difficult to determine a depth by which to cut the cornea. Too shallow a depth increases uncut portions of the cornea so that the cornea fails to be separated. Too great a depth can possibly make the laser reach living tissue and damage the human body.

### SUMMARY OF THE INVENTION

An aspect of the present invention is generally related to making it possible to easily find out a planned cutting depth of the cornea.

According to an aspect of the present invention, an ophthalmologic apparatus includes an obtaining unit configured to obtain an anterior segment image of a subject's eye, a tomographic image obtaining unit configured to obtain a tomographic image of an anterior segment of the subject's eye, a calculation unit configured to calculate a thickness of a cornea based on the tomographic image, and a display control unit configured to cause a display unit to display the anterior segment image, a closed curve on the anterior segment image, and a smallest thickness of the cornea among thicknesses of the cornea in a portion where the closed curve lies.

Further features and aspects of the present invention will become apparent from the following detailed description of exemplary embodiments with reference to the attached drawings.

### BRIEF DESCRIPTION OF THE DRAWINGS

The accompanying drawings, which are incorporated in and constitute a part of the specification, illustrate exemplary embodiments, features, and aspects of the invention and, together with the description, serve to explain the principles of the invention.

FIG. 1 is a schematic diagram illustrating an example of configuration of an ophthalmologic apparatus according to an exemplary embodiment.

FIG. 2 is a diagram illustrating an example of the structure of an eye.

FIG. 3 is a flowchart illustrating an example of a processing operation of the ophthalmologic apparatus according to the present exemplary embodiment.

FIG. 4 illustrates a display example of a display unit 192 according to the present exemplary embodiment.

FIG. 5 is a diagram illustrating an example of a method for selecting a pupil center.

FIG. 6 is a diagram illustrating an example of a line profile of cornea thickness.

### DESCRIPTION OF THE EMBODIMENTS

Various exemplary embodiments, features, and aspects of the invention will be described in detail below with reference to the drawings.

An apparatus according to an exemplary embodiment is applicable to a subject such as a subject's eye, skin, and visceral organs. Examples of the apparatus according to an exemplary embodiment include an ophthalmologic apparatus and an endoscope. As an example of an exemplary embodiment, an ophthalmologic apparatus according to the present exemplary embodiment will be described in detail below with reference to the drawings.

Overall Configuration of Apparatus

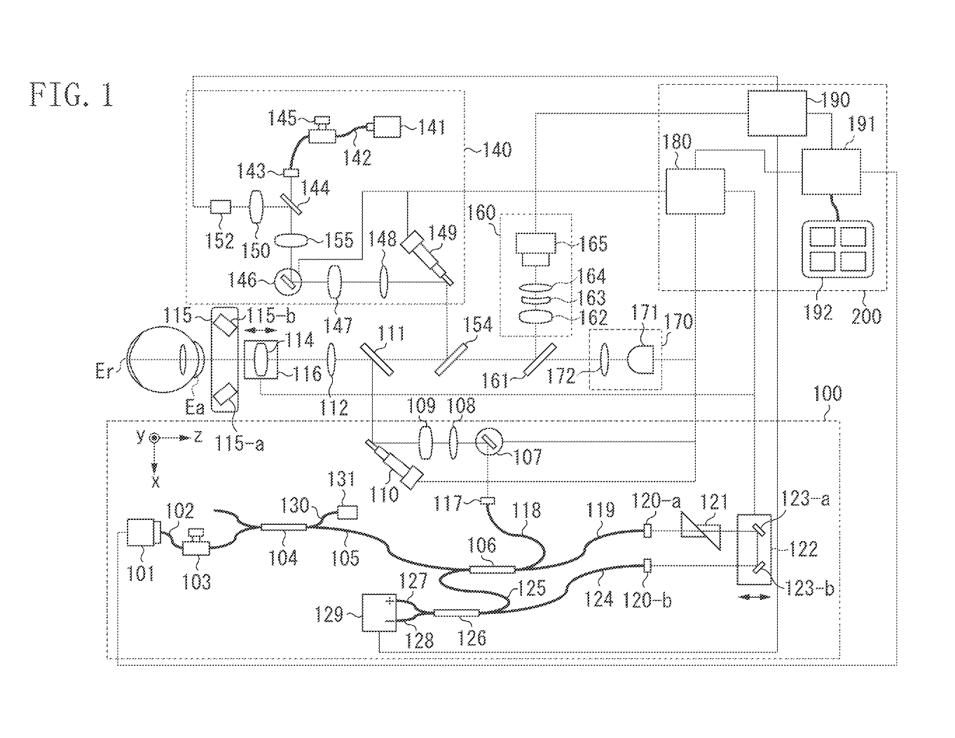

FIG. 1 is a schematic diagram illustrating an example of configuration of the ophthalmologic apparatus according to the present exemplary embodiment.

The present ophthalmologic apparatus includes a swept source optical coherence tomography (SS-OCT; hereinafter, may be referred to simply as an OCT) 100, a scanning laser ophthalmoscope (hereinafter, may be referred to as an SLO) 140, an anterior segment imaging unit 160, an internal fixation light 170, and a control unit 200. In another exemplary embodiment, the ophthalmologic apparatus need not include the SLO 140.

The internal fixation light 170 is lit to cause a subject's eye to gaze upon it. In that state, the anterior segment of the subject's eye is observed by the anterior segment imaging unit 160. The resulting image is used to perform alignment of the ophthalmologic apparatus. After the completion of the alignment, the fundus is imaged by the OCT 100 and the SLO 140.

Configuration of OCT 100

An example of configuration of the OCT 100 will be described.

The OCT 100 obtains tomographic images of the anterior segment of the subject's eye. In other words, the OCT 100 corresponds to an example of a tomographic image obtaining unit that obtains a tomographic image of the anterior segment.

A light source 101 is a variable wavelength light source. For example, the light source 101 emits light with a center wavelength of 1040 nm and a bandwidth of 100 nm. A control unit 191 controls the wavelength of the light emitted from the light source 101. More specifically, when obtaining a tomographic image, the control unit 191 sweeps wavelength of the light emitted from the light source 101. The control unit 191 corresponds to an example of a control unit that sweeps wavelength of the light emitted from the light source 101. The light emitted from the light source 101 is guided through a fiber 102 and a polarization controller 103 to a fiber coupler 104. In the fiber coupler 104, the light branches to a fiber 130 for light amount measurement and a fiber 105 for OCT measurement. A power meter (PM) 131 measures power of the light emitted from the light source 101 via the fiber 130. The light passed through the fiber 105 is guided to a second fiber coupler 106. In the fiber coupler 106, the light branches into measurement light (also referred to as OCT measurement light) and reference light.

The polarization controller 103 adjusts a polarization state of the light emitted from the light source 101 to linear polarization. The fiber coupler 104 has a branching ratio of 99:1. The fiber coupler 106 has a branching ratio of 90:10 (reference light to measurement light). The branching ratios are not limited to such values, and different values may be used.

The measurement light branched from the fiber coupler 106 is passed through a fiber 118 and emitted as parallel light from a collimator 117. The emitted measurement light reaches a lens 109 via an X scanner 107 and a lens 108. The X scanner 107 includes a galvanometer mirror for scanning the fundus Er in a horizontal direction (vertical direction in the diagram) with the measurement light. The measurement light from the lens 109 reaches a dichroic mirror 111 via a Y scanner 110. The Y scanner 110 includes a galvanometer mirror for scanning the fundus Er in a vertical direction (depth direction in the diagram) with the measurement light. A drive control unit 180 can control the X scanner 107 and the Y scanner 110 so that a desired range of area on the fundus Er is scanned with the measurement light. The dichroic mirror 111 has a characteristic of reflecting light of, for example, 950 nm to 1100 nm and transmitting other light.

The measurement light reflected by the dichroic mirror 111 passes through a lens 112 and reaches a focus lens 114 lying on a stage 116. The focus lens 114 focuses the measurement light on retinal layers of the fundus Er through the anterior segment Ea of the eye which is the subject. In other words, the optical system extending from the light source 101 to the subject's eye corresponds to an example of an illumination optical system that guides the light emitted from the light source 101 to the subject's eye. The measurement light irradiates the fundus Er to be reflected and scattered by the retinal layers and returns to the fiber coupler 106 through the foregoing optical path. The measurement light from the fundus Er reaches a fiber coupler 126 from the fiber coupler 106 through a fiber 125.

The drive control unit 180 controls movement of the focus lens 114 in the direction of the optical axis. In the present exemplary embodiment, the focus lens 114 is used by the OCT 100 and the SLO 140 in common. However, this is not restrictive. Different focus lenses may be provided for the respective optical systems. The drive control unit 180 may control the focus lens 114 by driving the focus lens 114 based on a difference between the wavelength the light source 101 uses and the wavelength a light source 141 uses. For example, if the focus lens 114 is provided for the OCT 100 and the SLO 140 in common, the drive control unit 180 moves the focus lens 114 according to a difference in wavelength when image capturing using the SLO 140 and image capturing using the OCT 100 are switched. If the optical systems of the OCT 100 and the SLO 140 include respective different focus lenses, when the focus lens of one optical system is adjusted, the drive control unit 180 moves the focus lens of the other optical system according to a difference in wavelength.

In an imaging mode for capturing a tomographic image of the anterior segment Ea, a focus position is adjusted to a predetermined portion of the anterior segment Ea, not to the fundus Er. The focus adjustment to the anterior segment Ea may be performed by moving the position of the focus lens 114. Alternatively, an optical member such as a dedicated lens may be inserted into an optical path before and/or after the focus lens 114 to adjust the focus position. Such an optical member can be detachably inserted into the optical path by a drive unit. When an anterior segment imaging mode is selected, the drive control unit 180 inserts the optical member into the optical path. When a fundus imaging mode is selected, the drive control unit 180 retracts the optical member from the optical path.

Meanwhile, the reference light branched from the fiber coupler 106 is passed through a fiber 119 and emitted as parallel light from a collimator 120-a. The emitted reference light is passed through dispersion compensation glass 121, reflected by mirrors 123-a and 123-b on a coherence gate stage 122, and passed through a collimator 120-b and a fiber 124 to reach the fiber coupler 126. The drive control unit 180 controls the coherence gate stage 122 to correspond to a difference in the eye axial length of the subject's eye.

Reaching the fiber coupler 126, the measurement light and the reference light are combined into interference light. The interference light travels through fibers 127 and 128 to reach a balanced receiver 129 which is an optical detector. The balanced receiver 129 converts an interference signal into an electrical signal. In other words, the optical system extending from the subject's eye to the balanced receiver 129 corresponds to an example of an imaging optical system that guides return light of light swept by a control unit, returning from the subject's eye, to an imaging unit. A signal processing unit 190 analyzes the converted electrical signal. Note that the optical detector is not limited to a balanced receiver, and other detectors may be used.

While the interference between the measurement light and the reference light occurs in the fiber coupler 126, such a configuration is not restrictive. For example, the mirror 123-a may be arranged to reflect the reference light to the fiber 119 so that interference between the measurement light and the reference light occurs in the fiber coupler 106. In such a case, the mirror 123-b, the collimator 120-b, the fiber 124, and the fiber coupler 126 become unnecessary. A circulator can be used here.

Configuration of SLO 140

An example of configuration of the SLO 140 will be described.

The SLO 140 corresponds to an example of a fundus image obtaining unit that obtains a fundus image of the subject's eye.

An example of the light source 141 is a semiconductor laser. In the present exemplary embodiment, the light source 141 emits light having a center wavelength of, for example, 780 nm. Measurement light (also referred to as SLO measurement light) emitted from the light source 141 is passed through a fiber 142, adjusted to linear polarization by a polarization controller 145, and emitted as parallel light from a collimator 143. The emitted measurement light passes through a perforated portion of a perforated mirror 144 and reaches a dichroic mirror 154 via a lens 155, an X scanner 146, lenses 147 and 148, and a Y scanner 149. The X scanner 146 includes a galvanometer mirror for scanning the fundus Er in the horizontal direction with the measurement light. The Y scanner 149 includes a galvanometer mirror for scanning the fundus Er in the vertical direction with the measurement light. The polarization controller 145 may be omitted. The drive control unit 180 can control the X scanner 146 and the Y scanner 149 so that a desired range of area on the fundus Er is scanned with the measurement light. The dichroic mirror 154 has a characteristic of reflecting light of, for example, 760 nm to 800 nm and transmitting other light.

The linearly-polarized measurement light reflected by the dichroic mirror 154 is transmitted through the dichroic mirror 111 to reach the fundus Er through a similar optical path to that of the OCT measurement light of the OCT 100.

The SLO measurement light irradiates the fundus Er to be reflected and scattered by the fundus Er and reaches the perforated mirror 144 through foregoing the optical path. The perforated mirror 144 reflects the light, which an avalanche photodiode (APD) 152 receives through a lens 150. The APD 152 converts the light into n electrical signal. The signal processing unit 190 receives the electrical signal.

The perforated mirror 144 is located in a position conjugate with a pupil position of the subject's eye. Part of the reflected and scattered light of the measurement light with which the fundus Er is irradiated passes through peripheral portions of the pupil. Such light is reflected by the perforated mirror 144.

Anterior Segment Imaging Unit 160

An example of configuration of the anterior segment imaging unit 160 will be described.

The anterior segment imaging unit 160 corresponds to an example of an obtaining unit that acquires an anterior segment image of the subject's eye.

The anterior segment imaging unit 160 includes lenses 162, 163, and 164, and an anterior segment camera 165.

An illumination light source 115 includes light-emitting diodes (LEDs) 115-a and 115-b. The LEDs 115-a and 115-b emit illumination light having a wavelength of, for example, 850 nm. The illumination light source 115 illuminates the anterior segment Ea. The light reflected by the anterior segment Ea reaches a dichroic mirror 161 through the focus lens 114, the lens 112, and the dichroic mirrors 111 and 154. The dichroic mirror 161 has a characteristic of reflecting light of, for example, 820 nm to 900 nm and transmitting other light. The anterior segment camera 165 receives the light reflected by the dichroic mirror 161 through the lenses 162, 163, and 164. The light received by the anterior segment camera 165 is converted into an electrical signal, which the signal processing unit 190 receives. The signal processing unit 190 generates an anterior segment image.

Internal Fixation Lamp 170

The internal fixation lamp 170 will be described.

The internal fixation lamp 170 includes a display unit 171 and a lens 172. An example of the display unit 171 is a plurality of LEDs arranged in a matrix. The drive control unit 180 performs control to change the position of an LED or LEDs to turn on according to a portion to be imaged. The light from the display unit 171 is guided to the subject's eye through the lens 172. For example, the display unit 171 emits light of 520 nm to display a desired pattern under the control of the drive control unit 180.

Control Unit 200

The control unit 200 will be described.

The control unit 200 includes the drive control unit 180, the signal processing unit 190, the control unit 191, and a display unit 192. Examples of the control unit 200 include a central processing unit (CPU) and a micro processing unit (MPU). The control unit 200 may be configured with a field programmable gate array (FPGA). The control unit 200 is connected to a not-illustrated input unit, such as a mouse and a keyboard, by a wired or wireless connection. In the present exemplary embodiment, for example, the mouse includes sensors that detect movement signals when the examiner manually moves the mouse body in two dimensions, and two right and left mouse buttons for detecting depression by the examiner's hand. The mouse further includes a wheel mechanism that is rotatable in a front-back direction. The wheel mechanism is arranged between the two right and left mouse buttons. The control unit 200 is connected to a not-illustrated storage unit, such as a memory, by a wired or wireless connection. For example, the control unit 200 executes a program stored in the storage unit to perform various functions.

The drive control unit 180 controls various units as described above.

The signal processing unit 190 generates an image, analyzes the generated image, and generates visualization information about the analysis result based on respective signals output from the balanced receiver 129, the APD 152, and the anterior segment camera 165. The image generation will be described in detail below.

The control unit 191 controls the entire ophthalmologic apparatus, and displays an image generated by the signal processing unit 190 on a display screen of the display unit 192. The display unit 192 corresponds to an example of a display unit or display apparatus. Image data generated by the signal processing unit 190 may be transmitted to the control unit 191 in a wired or wireless manner.

An example of the display unit 192 is a liquid crystal display. The display unit 192 displays various types of information as will be described below under the control of the control unit 191. Image data from the control unit 191 may be transmitted to the display unit 192 in a wired or wireless manner. While in the present exemplary embodiment, the display unit 192 is included in the control unit 200, in another exemplary embodiment, the display unit 192 may be provided separate from the control unit 200.

The control unit 191 and the display unit 192 may be integrally configured as a tablet, which is an example of a user-portable apparatus. In such a case, the display unit 192 can include a touch panel function so that operations such as moving a display position of an image, scaling, and changing an image to display can be performed on the touch panel. The display unit 192 may include a touch panel function even if the control unit 191 and the display unit 192 are not integrally configured. In other words, a touch panel may be used as the input unit.

Image Processing

Next, the image generation and image analysis by the signal processing unit 190 will be described.

Generation of Tomographic Image and Generation of Fundus Image

The signal processing unit 190 generates a tomographic image by performing typical reconstruction processing on the interference signal output from the balanced receiver 129.

The signal processing 190 initially removes fixed pattern noise from the interference signal. For example, the signal processing unit 190 removes fixed pattern noise by averaging a detected plurality of A-scan signals to extract the fixed pattern noise and subtracting the fixed pattern noise from the input interference signal.

Next, the signal processing unit 190 performs desired window function processing to optimize depth resolution and a dynamic range, which have a tradeoff relationship there between when Fourier-transformed in a finite interval. The signal processing unit 190 then performs fast Fourier transform (FFT) processing to generate a tomographic image.

The OCT 100 can capture a tomographic image over a wider range (with a greater lateral dimension) than a spectral domain OCT (SD-OCT). The reason is as follows: With an SD-OCT spectroscope, the diffraction grating produces a loss of interference light. In contrast, the SS-OCT uses no spectroscope. For example, the SS-OCT can be configured to differentially detect interference light, which facilitates improving sensitivity. The SS-OCT having sensitivity equivalent to that of the SD-OCT can be made faster, and the faster speed can be utilized to obtain a tomographic image over a wider angle of view.

The OCT 100 can capture a tomographic image with a greater depth in the depth direction (with a greater vertical dimension) than the SD-OCT. The reason is as follows: With the SD-OCT spectroscope, the diffraction grating spatially disperses the interference light. As a result, the interference light easily produces crosstalk between adjoining pixels of a line sensor. Interference light from a reflection surface lying at a depth position Z=Z0 oscillates at a frequency of Z0/π with respect to a wave number k. The greater the Z0 (the greater the distance from a coherence gate position), the higher the oscillation frequency of the interference light and the greater the effect of the crosstalk of the interference light between adjoining pixels of the line sensor. Consequently, the SD-OCT drops significantly in sensitivity when imaging deeper positions. On the other hand, the SS-OCT using no spectroscope is advantageous in capturing a tomographic image in deeper positions as compared to the SD-OCT. The light source used in the SS-OCT has a wavelength longer than that of the light source used in the SD-OCT. The longer wavelength also contributes to the obtaining of a tomographic image deeper in the depth direction.

When the control unit 192 displays a tomographic image on a display area of the display unit 192, it is no use displaying an area that includes no own image of a tomographic image. In the present exemplary embodiment, when displaying a tomographic image, the control unit 191 identifies a portion that includes an own image of a tomographic image from data loaded in a memory in the signal processing unit 190. The control unit 191 cuts the tomographic image to the size of the display area, and displays the resulting tomographic image. An own image of a tomographic image refers to an image of the fundus Er or anterior segment tissue of the subject's eye.

Segmentation

The signal processing unit 190 performs segmentation of a tomographic image by using a luminance image.

The signal processing unit 190 initially applies a median filter and a Sobel filter to a tomographic image to be processed to create images (hereinafter, respectively referred to as a median image and a Sobel image). Next, the signal processing unit 190 creates profiles for respective A-scans from the created median image and Sobel image. The signal processing unit 190 creates luminance value profiles from the median image, and gradient profiles from the Sobel image. The signal processing unit 190 then detects peaks in the profiles created from the Sobel image. The signal processing unit 190 extracts boundaries between the respective areas of the retinal layers by referring to the profiles of the median image corresponding to before, after, and between the detected peaks.

The signal processing unit 190 further measures the thicknesses of the respective layers in the direction of the A-scan lines to create thickness maps of the layers. The signal processing unit 190 may only measure the thickness of the cornea and create a thickness map of the cornea without measuring the thickness of each layer. In other words, the signal processing unit 190 corresponds to an example of a calculation unit that calculates the thickness of the cornea based on a tomographic image.

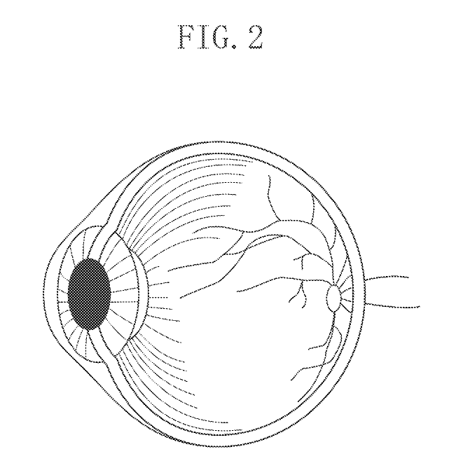

FIG. 2 illustrates the structure of a human eye. For example, the eyeball has a diameter of approximately 24 mm. The interior of the eyeball is filled with the vitreous body. The transparent cornea lays at the foremost surface of the eye, behind which are the iris and the pupil in a fundus direction. The crystalline lens follows. Light passed through the pupil forms an image on the retina inside the eyeball. The incident light forms an image on the retina because of refraction from the cornea and the crystalline lens. With respect to the refractive index of air of 1.0, the cornea has a refractive index of approximately 1.38 and the crystalline lens approximately 1.42. Such refractive indexes are just an example, and the present exemplary embodiment is not limited thereto. The cornea can be clouded due to aging, injuries, and diseases. The clouded cornea scatters light, and the light fails to form an accurate image on the retina. Corneal transplantation is performed to regenerate the cornea.

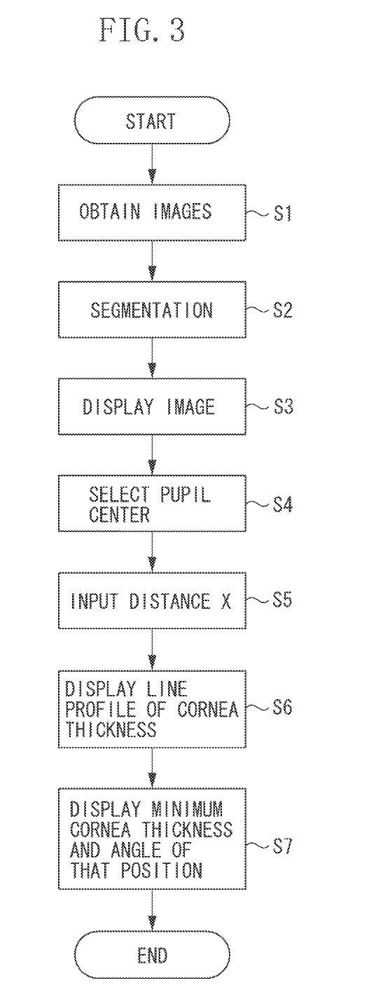

FIG. 3 is a flowchart illustrating an example of a processing operation of the present ophthalmologic apparatus.

In step S1, the ophthalmologic apparatus makes various adjustments of alignment and a coherence gate or the like, and then obtains tomographic images of the anterior segment of the subject's eye. Specifically, the signal processing unit 190 generates tomographic images of the anterior segment based on signals obtained by the OCT 100. The control unit 191 obtains the tomographic images of the anterior segment obtained by the OCT 100 radially scanning or raster scanning the anterior segment with the OCT measurement light, and obtains volume data (three-dimensional data) on the cornea from the tomographic images of the anterior segment. In other words, the control unit 191 corresponds to an example of the tomographic image obtaining unit that obtains a tomographic image of the anterior segment.

The signal processing unit 190 further generates a front image of the cornea from signals obtained by the anterior segment imaging unit 160. The control unit 191 obtains the front image of the cornea generated by the signal processing unit 190. In other words, the control unit 191 corresponds to an example of the obtaining unit that obtains an anterior segment image of the subject's eye. The above operations are performed in step S1.

The volume data can be obtained from a range where the entire cornea is seen. A circular range with a radius of 8 mm is employed for radial scanning. A square range of 16 mm on a side is employed for raster data. Since an image of the entire cornea needs to be obtained, the use of the SS-OCT is advantageous. The dimensions of the scan ranges are not limited to the foregoing, and may be changed as appropriate.

In the example described above, when obtaining the front image, the anterior segment imaging unit 160 captures the anterior segment image of the subject's eye coaxially almost simultaneously with the image capturing by the OCT 100. However, this is not restrictive. The signal processing unit 190 may reconstruct volume data obtained by using the OCT 100 to generate an anterior segment image (front image). Since the tomographic images and the anterior segment image are coaxially obtained generally at the same time, a correspondence relation can be established.

In step S2, the signal processing unit 190 performs segmentation of the tomographic images. The signal processing unit 190 initially applies a median filter and a Sobel filter to the tomographic images to be processed to create images (hereinafter, referred to as a median image and a Sobel image). Next, the signal processing unit 190 creates profiles for respective A-scans from the created median images and Sobel images. The signal processing unit 190 creates luminance value profiles from the median images, and gradient profiles from the Sobel images. The signal processing unit 190 detects peaks in the profiles created from the Sobel images. The signal processing unit 190 extracts the front and rear surfaces of the cornea by referring to the profiles of the median images corresponding to before, after, and between the detected peaks.

The signal processing unit 190 further measures thicknesses of the respective layers in the direction of the A-scan lines and creates thickness maps of the respective layers.

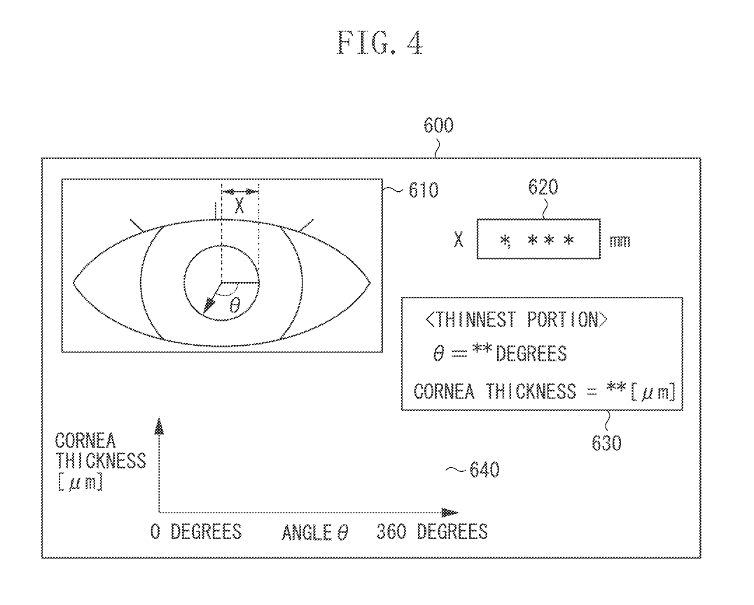

In step S3, the control unit 191 displays the anterior segment image on the display unit 192 as illustrated in FIG. 4. FIG. 4 is a diagram illustrating a display example 600 of the display unit 192. Specifically, the control unit 191 displays an anterior segment image 610, an interface 620, a line profile 640 of the thickness of a cornea cutting surface (hereinafter, referred to simply as a line profile 640), and information about a thinnest portion of the cornea 630. The interface 620 specifies a distance X from a pupil center to a position where the cornea is planned to be cut. The position where the cornea is planned to be cut may be referred to simply as the cutting position of the cornea. The anterior segment image displayed on the display unit 192 is the image obtained in step S1. In the example illustrated in FIG. 4, the interface 620, the line profile 640 of the thickness of the cornea cutting surface, and the information about the thinnest portion of the cornea 630 are illustrated only by their display frames without data input or displayed. While the display example 600 illustrated in FIG. 4 includes the anterior segment image 610, the interface 620, the information about the thinnest portion of the cornea 630, and the line profile 640, the items to be displayed are not limited thereto. For example, any of the pieces of information illustrated in FIG. 4 may be selected and displayed. Other information may be further displayed. The layout of the information displayed on the display unit 192 is not limited to the example illustrated in FIG. 4. Other layouts may be employed.

The anterior segment image 610 is an image generated by the signal processing unit 190 based on signals obtained by the anterior segment imaging unit 160. The interface 620 is an area for accepting an input from the examiner through the input unit. In other words, the control unit 191 displays an area for accepting an input from the input unit on the display unit.

The information about the thinnest portion of the cornea 630 is information about a portion where the cornea is thinnest in a cornea position specified by the examiner. For example, the information 630 includes information indicating the position where the cornea is thinnest and the value of the smallest cornea thickness. An example of the information indicating the position where the cornea is thinnest is an angle that a line connecting the pupil center and the position where the cornea is thinnest forms with respect to a horizontal line passing through the pupil center. While a clockwise angle is displayed in FIG. 4, a counterclockwise angle may be displayed. Such an angle and the thickness of the thinnest portion are determined by the signal processing unit 190, and displayed on the display unit 192 by the control unit 191. The information about the thinnest portion of the cornea 630 is not limited thereto, and may include a position where the cornea is the second thinnest and the thickness of the cornea. The line profile 640 is a graph illustrating the thickness of the cornea in a cornea position specified via the input unit. In the example illustrated in FIG. 4, the graph displays thicknesses of the cornea at respective cornea positions indicated by angles.

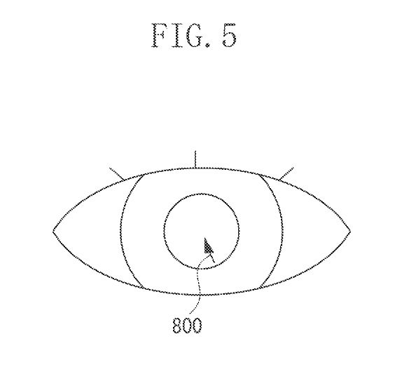

In step S4, the examiner manually selects a pupil center on the anterior segment image 610 by using the input unit such as a mouse and a touch panel. The control unit 191 displays the selected point as the pupil center on the anterior segment image 610 on the display unit 192. FIG. 5 is a diagram illustrating a method for manually selecting the pupil center. Specifically, FIG. 5 illustrates a case where a cursor 800 moves in response to an input to the input unit and the examiner selects the position of the cursor 800 as the pupil center. The pupil center need not necessarily be manually selected. The pupil center may be extracted by image processing. Specifically, the signal processing unit 190 can binarize the anterior segment image to detect the pupil, and determine the center of gravity of the detected pupil to extract the pupil center. In the present exemplary embodiment, the pupil center is not a concept that covers only the perfect center of the pupil, but one that covers a general center of the pupil.

In step S5, having selected the pupil center, the examiner inputs a distance X from the pupil center to a planned cutting position of the cornea to the interface 620 via the input unit. A circle with a radius X about the pupil is the planned cutting position of the cornea. The control unit 191 displays the cir le with the radius X about the position selected in step S4 as superimposed on the anterior segment image 610. In other words, the control unit 191 corresponds to an example of a display control unit that causes a display unit to display an anterior segment image and, on the anterior segment image, a closed curve indicating the cutting position of the cornea of the subject's eye. The control unit 191 also causes the closed curve to be displayed on the anterior segment image 610 in a size according to the input to the interface 620 (area).

For example, in the present exemplary embodiment, the examiner inputs a distance X of 3.5 mm. The input distance X is not limited to such a value, and other values may be input. Steps S4 and S5 may be performed in a reverse order.

In step S6, the control unit 191 displays on the display unit 192 a line profile 640 of the cornea thickness along the circle with the radius X displayed on the anterior segment image 610 in step S5. In other words, the control unit 191 causes the display unit to display a graph that indicates thicknesses of the cornea in the portion where the closed curve lies with respect to positions on the closed curve. Cornea thicknesses along the circle with the radius X are determined by the signal processing unit 190.

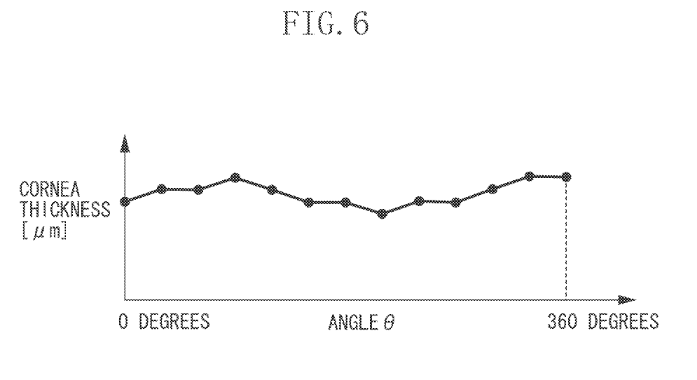

The vertical axis of the line profile 640 indicates the cornea thickness. The horizontal axis indicates an angle θ that is expressed with reference to the pupil center. FIG. 6 is a diagram illustrating an example of the line profile 640 of the cornea thickness.

After the determination of the cornea thicknesses along the circle with the radius X, the control unit 191 performs step S7. In step S7, the control unit 191 causes the display unit 192 to display a minimum cornea thickness among the cornea thicknesses along the circle with the radius X and an angle of that position as the information about the thinnest portion of the cornea 630. In other words, the control unit 191 causes the display unit to display a smallest cornea thickness among the cornea thicknesses in the portion where the closed curve lies. The control unit 191 further causes the display unit to display a display pattern that indicates the position where the cornea has the smallest thicknesses among the thicknesses of the cornea in the portion where the closed curve lies.

The control unit 191 causes an arrow that indicates the position where the cornea thickness becomes minimum on the circle with the radius X to be displayed on the anterior segment image 610. The display indicating the position where the cornea thickness becomes minimum on the circle with the radius X is not limited to the arrow illustrated in FIG. 4. Other display patterns may be used as long as the position where the cornea thickness becomes minimum is indicated. In other words, the control unit 191 causes a display pattern that indicates the position where the cornea has the smallest thickness among the cornea thicknesses in the portion where the closed curve lies to be displayed on the anterior segment image 610.

The information about the thinnest portion of the cornea 630 may be displayed before the display of the line profile 640.

According to the present exemplary embodiment, the examiner can thus check on the anterior segment image 610 the depth of the position where to cut the cornea.

A femtosecond laser may be used concurrently with operations other than corneal transplantation. Examples include refractive surgery. In such a concurrent use, it may take long from cornea cutting to an operation because the femtosecond laser apparatus is placed in a location physically remote from the operating room. If it takes long from the cornea cutting using the femtosecond laser to the operation, a process for infection prevention is needed. The process includes not completely cutting off the cornea with the femtosecond laser but cutting the cornea as much as a thickness not to penetrate through the cornea, for example, by a depth 90% that of the thinnest portion of the cornea cutting surface (precutting), and completely cutting off the cornea with a diamond knife immediately before the operation. The thickness not to penetrate through the cornea depends on the thickness of the cornea of the subject's eye. The examiner then examines the cornea thickness of the subject's eye by using a tomographic image apparatus such as an anterior segment OCT, and sets the cutting depth of the precutting to, for example, a thickness 90% that of the thinnest portion of the cornea cutting surface. An elevation map can display cornea thicknesses by color, but not in concrete figures. It has therefore been difficult to accurately find out how thick to precut the cornea.

According to the present exemplary embodiment, the ophthalmologic apparatus displays a smallest cornea thickness in the planned cutting position of the cornea. As a result, the examiner can easily find out how thick to precut the cornea.

An SD-OCT, for example, has a narrower angle of view than an SS-OCT. The SD-OCT may fail to obtain a tomographic image at a specified planned cutting position of the cornea (for example, a position at a radius of 3.5 mm) and/or fail to obtain a sharp image more often than the SS-OCT.

The SS-OCT can obtain tomographic images at higher speed and with a wider angle of view than the SD-OCT. The SS-OCT is therefore less susceptible to the movement of the subject's eye and the restriction of the angle of view, and can accurately and reliably display cornea thicknesses at specified positions like a planned cutting position of the cornea with a radius of 3.5 mm from the pupil. Since the ophthalmologic apparatus determines cornea thicknesses from tomographic images obtained by the SS-OCT, which are sharp in the depth direction, the examiner can more accurately find out the smallest thickness of the cornea.

A femtosecond laser can cut the cornea in a desired position based on control information from outside. Data on the line profile of the cornea thickness obtained in the present exemplary embodiment can be input to the femtosecond laser to cut the cornea in an optimum depth with an arbitrary cornea cutting surface. The examiner shall input the values of the cornea thicknesses included in the data on the line profile of the cornea thickness obtained in the present exemplary embodiment, multiplied by 0.9, to the femtosecond laser as setting values of the cutting depth.

Other Embodiments

The present invention is not limited to the foregoing exemplary embodiment. Any other exemplary embodiments that would enable practice of the present disclosure are applicable.

For example, in the foregoing exemplary embodiment, the examiner inputs the distance X in step S5 before the control unit 191 displays a circle with the radius X on the anterior segment image 610. This is not restrictive. For example, when the pupil center is selected in step S4, the control unit 191 may automatically display a circle with a predetermined radius X on the anterior segment image 610. An initial value of the radius X may be arbitrarily set, like 3.5 mm. Such a setting may allow the examiner to omit inputting a radius X. The radius of 3.5 mm (diameter of 7 mm) is a frequently used value in corneal transplantation. Using such a value as the initial value increases the possibility that the examiner can omit inputting a radius X.

The examiner can change the radius X by inputting a radius X into the interface 620. However, this is not restrictive. For example, when the examiner puts the cursor 800 on the anterior segment image 610 and rotates the mouse wheel, the control unit 191 may change the radius X according to the direction of rotation. This facilitates changing the radius X. Alternatively, to facilitate changing the radius X, the ophthalmologic apparatus may be configured to change the value of the radius X by the examiner dragging the circle of the radius X while displaying thereof on the anterior segment image 610.

The foregoing exemplary embodiment uses the SS-OCT. This is not restrictive, and a time domain OCT (TD-OCT) or an SD-OCT may be used.

In the foregoing exemplary embodiment, the OCT 100 is used to obtain tomographic images of the cornea in the anterior segment Ea. This is not restrictive, and a Scheimpflug camera may be used. The anterior segment imaging unit 160 is used to obtain the anterior segment image 610. This is not restrictive, and the SLO 140 may be used to obtain an anterior segment image.

In the foregoing exemplary embodiment, the planned cutting position of the cornea is circular. This is not restrictive, and any closed curve may be used. To display a closed curve other than a circle, the examiner specifies a plurality of points on the anterior segment image 610 by using the input unit. The control unit 191 displays a curve connecting the points.

In the foregoing exemplary embodiment, it is the radius X that the examiner inputs from the input unit. This is not restrictive, and the examiner may input a diameter.

In the foregoing exemplary embodiment, the ophthalmologic apparatus displays the value of the thinnest portion of the cornea. This is not restrictive, and the ophthalmologic apparatus may display a precut value. For example, the ophthalmologic apparatus multiplies the value of the thinnest portion by 0.9 and displays the resulting precut value. The signal processing unit 190 performs the multiplication processing. The precut depth need not be 90% that of the thinnest portion. If a different value is used, the multiplier is also changed.

In the foregoing exemplary embodiment, the planned cutting position of the cornea is determined based on the pupil center. This is not restrictive, and the planned cutting position of the cornea may be determined based on the position of a apex of the cornea. More specifically, the closed curve displayed by the display control unit is a circle around the center of the pupil of the subject's eye or around the apex of the cornea.

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-082688, filed Mar. 30, 2012, which is hereby incorporated by reference herein in its entirety.

What is claimed is:

1. An ophthalmologic apparatus comprising:
an obtaining unit configured to obtain an anterior segment image of a subject's eye;
a tomographic image obtaining unit configured to obtain a tomographic image of an anterior segment of the subject's eye;
a calculation unit configured to calculate a thickness of a cornea based on the tomographic image; and
a display control unit configured to cause a display unit to display the anterior segment image, a closed curve on the anterior segment image, and a smallest thickness of the cornea among thicknesses of the cornea in a portion where the closed curve lies.

2. The ophthalmologic apparatus according to claim 1, wherein the closed curve is a circle around a center of a pupil of the subject's eye or around an apex of the cornea.

3. The ophthalmologic apparatus according to claim 1, wherein the display control unit is configured to cause the display unit to display a display pattern to indicate a position where the cornea has the smallest thickness among the thicknesses of the cornea in the portion where the closed curve lies.

4. The ophthalmologic apparatus according to claim 1, wherein the display control unit is configured to cause a display pattern indicating a position where the cornea has the smallest thickness among the thicknesses of the cornea in the portion where the closed curve lies to be displayed on the anterior segment image.

5. The ophthalmologic apparatus according to claim 1, wherein the tomographic image obtaining unit comprises:
an illumination optical system configured to guide light emitted from a light source to the subject's eye;
a control unit configured to sweep wavelength of the light emitted from the light source;
an imaging optical system configured to guide return light of the light swept by the control unit, returning from the subject's eye, to an imaging unit; and
a tomographic image generation unit configured to generate the tomographic image based on an output of the imaging unit.

6. The ophthalmologic apparatus according to claim 1, wherein the display control unit is configured to cause the display unit to display a graph indicating the thicknesses of the cornea in the portion where the closed curve lies with respect to positions on the closed curve.

7. The ophthalmologic apparatus according to claim 1, wherein the display control unit is configured to cause the display unit to display an area accepting an input from an input unit, and cause the closed curve to be displayed on the anterior segment image in a size according to the input to the area.
